(19) United States
(12) Patent Application Publication
(10) Pub. No.: US 2010/0100138 A1
(43) Pub. Date: Apr. 22, 2010

(54) Endoscopic Insturments and Mehod for Delivery of Spinal Implant

(76) Inventors: Joseph E. Reynolds, Cincinnati, OH (US); Eric J. Wall, Cincinnati, OH (US); Alvin H. Crawford, Cincinnati, OH (US)

Correspondence Address: BAKER & DANIELS LLP, 300 NORTH MERIDIAN STREET, SUITE 2700, INDIANAPOLIS, IN, 46204, US

(21) Appl. No.: 11/992,463

(22) PCT Filed: Sep. 21, 2006
(86) PCT No.: PCT/US06/36884

§ 371 Date: Dec. 14, 2009

Related U.S. Application Data
(60) Provisional application No. 60/719,076, filed on Sep. 21, 2005.

(51) Int. Cl. A61B 17/56; A61F 2/44

(52) U.S. Cl. 606/86 A; 623/17.11

(57) ABSTRACT

Instruments and methods for the installation of spinal implants (10, 10′, 10″). Osteotomes (50, 74, 90, 150) are provided for establishing a datum and pre-cutting the vertebra (30) and covering tissue. Insertion instruments (100, 100′, 200) are provided for holding, aligning, placing, and inserting the spinal implant (10, 10′, 10″).

### CROSS-REFERENCE TO RELATED APPLICATION

U.S. Provisional Patent Application Ser. No. 60/719,076, filed Sep. 21, 2005, is expressly incorporated by reference herein.

### BACKGROUND AND SUMMARY OF ILLUSTRATIVE EMBODIMENTS

The current invention relates generally to instruments and methods for installation of spinal implants and, more specifically, to implants used in the correction, arresting or slowing of abnormal curvature of the spine, including scoliosis, hyperlordosis and hypokyphosis.

Spinal correction systems may include a spinal implant such as a staple, similar to that described in U.S. Pat. No. 6,746,450 Wall et al. and U.S. patent application Ser. No. 11/126,782, filed May 11, 2005, the disclosures of which are expressly incorporated by reference herein. It is often desirable to install such spinal implants endoscopically to reduce trauma, blood loss, operating room (OR) time, pain, recovery time, and cost. Endoscopic installation of spinal implants requires good visibility, and accurate placement. It is often desirable to precut the bone to allow easy placement without fear of splitting or cracking the vertebra. It also is desirable to place the implant with a limited number of steps. Accuracy of placement and insertion is important in all spinal implants including those using hemiepiphysiodesis principles. Proper placement is necessary to provide an appropriate pattern of growth plate compression force distribution and to avoid disrupting the disc or growth plates during installation.

Endoscopic insertion of spinal implants typically requires placement of fasteners such as screws or anchors. Such installation in vertebral bone is often accomplished via impacting the device to drive it into the bone. Methods are needed to insert the implant in bone to reduce the potential for splitting or over driving the fastener or implant.

As noted above, installation of a spinal implant can require impacting the implant (or the application of other forms of energy) to drive it into the bone. Often impact or other energy applications can result in sticking or wedging of the associated insertion instrument, such that significant force is required to disengage the instrument from the implant. Methods are needed for disengaging the instrument after impact, or avoiding impact altogether. Illustratively, the insertion instrument should disengage from the implant device following installation without loosening or dislodging the implant.

Illustratively, instrumentation and methods are desired to:
1. Allow endoscopic installation and allow access through an endoscopic port;
2. Prevent over penetration of a guide wire and/or other locating feature(s);
3. Improve the ease of placement and reduce the number of steps required for planning, pre-cutting the bone, implant insertion, and fastener installation (e.g. screws);
4. Accurately place the implant to avoid damaging the disc or growth plate;
5. Disengaging the instrument from the implant following insertion;
6. Precut the bone for easy placement and reduced force required for insertion of the implant device;
7. Insert the implant without impact such as using other forms of reciprocating energy;
8. Precut fastener holes for accuracy, ease of placement, assure proper angle, and assure the fastener follows the preplanned path for entry in the bone; and/or
9. Simplify engagement (attachment) and disengagement of the instrument from the implant and assure that after placement the implant will not be dislodged during detachment and removal of the instrument.

### Illustrative Osteotomes for Pre-Cutting of the Bone (Chisel Tools)

An endoscopic or open surgical instrument, or osteotome, is illustrated for precutting the vertebra and tissue covering the vertebra (e.g., the pleura) for placement of an orthopedic medical implant device such as a staple. A first illustrative embodiment osteotome has a sharp needle that is used as a datum for planning the pre-placement and alignment prior to cutting. The needle is intended to pierce the disc and mark the location of the hole for future reference. Imaging (such as radiography or fluoroscopy) may be used for planning and to assure proper placement prior to cutting the bone. The illustrative osteotome has features for pre-cutting the tissue and bone for the spinal implant, including staple leg(s) and other features such as an anti-rotation or stabilization members. Cutting is illustratively executed by impacting the handle, or applying an energy source such as ultrasonic energy or reciprocating motion. The datum needle or the area surrounding it may be partly or entirely coated with a dye, such as methyl blue, to mark the location of the datum hole created by the needle for future reference.

In a further illustrative embodiment, a plurality of osteotomes are provided in sizes equivalent to the available sizes of a plurality of spinal implants. The plurality of osteotomes and spinal implants may be provided as a surgical kit, wherein the osteotomes are used to select the appropriate implant size prior to cutting. The appropriate sized osteotome is placed adjacent the spine and the position checked prior to cutting. The osteotomes may be made of an optically opaque material (such as stainless steel or other metals) to assure they are visible under imaging, such as fluoroscopy. The peripheral dimensions of the planar profile of the osteotomes are illustratively the same as the peripheral dimensions of the anterior-posterior planar profile of the implant, thereby allowing the surgeon to assure that both the size and placement are correct prior to cutting. Once size and position are confirmed, the osteotome is taped down (impacted) to pre-cut the bone and tissue overlying the vertebral body or bone. The osteotome illustratively pre-cuts incisions for legs of the implant and simultaneously cuts holes for fasteners or other features. More particularly, the osteotome is configured to cut through overlying tissue as well as the compact or cortical bone (outer layer) of the vertebrae. The cuts and holes (or incisions) act as a datum to mark and guide the implant to the exact location selected for placement of the spinal implant.

### Illustrative Insertion Instruments (Placement Tools)

An endoscopic or open insertion instrument is illustrated for holding, aligning, placing, and inserting an orthopedic medical implant device such as a spinal implant or staple. The insertion instrument illustratively has one or more awl features for pre-cutting fastener holes or other features that may be needed for placement of fasteners (such as bone screws) for the spinal implant. The insertion instrument illustratively includes a blunt wire or pin that is used as a datum or locator for alignment of the spinal implant. Optionally, the awls could be included with the osteotomes detailed herein.

When the insertion instrument is used with the first illustrative embodiment osteotome detailed above, a blunt wire or pin may be inserted in the original hole made by the osteotome needle. The location of the hole may be easier to find when using the optional marking discussed above. The blunt wire or pin becomes a datum to assure alignment of the spinal implant to the precut bone.

Additional illustrative embodiment endoscopic or open insertion instruments may be provided for holding, aligning, placing, and inserting orthopedic medical implant devices such as spinal implants or staples. Again, these insertion instruments may be used with osteotomes to pre-cut holes for fasteners or other features. The insertion instrument may include a blunt wire or pin that is used as a datum or locator for alignment of the spinal implant.

In certain illustrative embodiments, the insertion instrument allows the fastener, illustratively bone screws, to be pre-assembled in the spinal implant to eliminate the added steps of placing the fasteners as a part of the surgical procedure. The screws may also be utilized to hold or attach the spinal implant to the instrument and to allow for easy disengagement (or detachment) following placement.

A further illustrative embodiment insertion instrument for the holding, aligning, placement, and insertion of a spinal implant or staple is disclosed, wherein the instrument may also be used for extraction or removal of the implant, as required. The instrument is useful in both endoscopic or open procedures. Alignment and placement may be accomplished by positioning the staple in the incisions or cuts made by one of the illustrative embodiment osteotomes detailed herein. In one illustrative embodiment, the insertion instrument and osteotome are designed without the need for a needle, blunt wire, or pin for use as a datum or locator for alignment of the spinal implant or staple as previously disclosed.

This illustrative embodiment insertion instrument also allows the fasteners, illustratively bone screws, to be pre-assembled in the implant to eliminate the added steps of placing the fasteners as a part of the surgical procedure, thereby reducing the time in surgery and reducing the potential of dropping a screw in the body cavity. While the security and stability of the screws during handling and manipulation may be achieved by a variety of means, one illustrative embodiment utilizes a close fit between the screw threads and the internal threads of the implant. The interference can be easily overcome when the screws are placed or rotated into the vertebral bone.

### Reciprocating Motion or Ultrasonic Insertion

Ultrasonic and reciprocating cutting and coagulation devices are well known in the art. In a further illustrative embodiment insertion instrument, a reciprocating motor or piezoelectric horn suited for minimally invasive surgery is included in the handle, wherein a shaft extends from the motor for transmitting ultrasonic energy to the spinal implant which is operably coupled to the shaft. The motor may comprise a piezoelectric transducer that produces reciprocating motion of the shaft, but is not limited by the type of motor. The motor may be any type of actuator that can produce the required energy or reciprocating motion. The frequency of the reciprocation or vibration illustratively varies from any frequency above approximately 1 kHz, but is preferably set at the natural frequency of the spinal implant which will reduce the power required for driving the reciprocation (e.g., about 10 to 20 kHz for the illustrative embodiment spinal implant).

The illustrative embodiment spinal implant or staple tends to flex about an off set centerline such that the two leg tips of the implant vibrate toward and away from each other. The offset or angle of the center of flex is related to the center of gravity about this axis. As discussed, this type of energy can also be applied to pre-cutting the overlying tissue and the bone prior to insertion of the spinal implant.

### Articulation

Each of the illustrative insertion instruments may be optionally articulated or hinged to facilitate insertion through a port. The articulation allows the spinal implant to be articulated preferably 90-degrees to allow the implant to pass through the port at its narrowest attitude. Similarly, the illustrative osteotomes may be articulated, or the implants themselves may articulate or fold to permit easy insertion.

### Illustrative Methods

The present disclosure further includes methods of operation related to the illustrative surgical instruments detailed herein. For example, methods are disclosed for establishing a datum of an anatomical structure such as a spinal disc, precutting incisions in tissue and/or bone, and using the needle hole for placement of a medical device in the precut bone or tissue incision(s). Moreover, the illustrative methods facilitate accurate placement of the legs or blades of a spinal implant by using a reference datum. In one illustrative method, a separate blunt wire or pin is used to locate a marked needle hole and align the implant for placement. The length of the blunt wire or pin is limited to assure it does not protrude through the anatomical structure (disc) and hit other structures (blood vessels, nerves or other anatomical structures).

An illustrative insertion method includes of the following steps:
1. Planning placement of the implant, inserting a short needle to establish a hole as a datum, and optionally checking the location using imaging such as radiography;
2. Inserting the spinal correction implant in the bone using mechanical energy (impact force, ultrasonic or sonic energy, reciprocating motion, or other). Simultaneously inserting one or more awl(s) to precut holes for placement of bone screws or other fasteners, where applicable; and
3. Placing and inserting fasteners, where applicable.

An illustrative precutting insertion method includes of the following steps:
1. Planning placement of the implant using a template or osteotome and optionally checking the location using imaging as radiography;
2. Using a sharp, relatively short needle to establish a datum hole and optionally marking the hole for easy identification;
3. Pre-cutting the overlying tissue (if any) and at least the surface of the bone using mechanical energy (impact force, ultrasonic energy, or reciprocating motion or other);
4. Locating the needle hole and inserting a blunt wire or pin (illustratively, the needle hole was marked in step 2 above for easy location);
5. Using the blunt wire or pin as a datum for locating and placing the spinal correction implant;
6. Inserting the spinal correction implant in the bone using mechanical energy (impact force, ultrasonic or sonic energy, reciprocating motion, or other) and simultaneously inserting one or more awl(s) to precut holes for placement of bone screws or other fasteners; and
7. Inserting fasteners or screws in the holes cut by the awl(s).

A further illustrative method includes the following additional steps:
1. Inserting the spinal correction implant in the bone using mechanical energy (impact force, ultrasonic or sonic energy, reciprocating motion, or other) and simultaneously inserting one or more bone screws or other fasteners; and
2. Disengaging the insertion instrument from the screws and individually tightening each screw.

A further illustrative insertion method includes the steps of planning and pre-cutting the bone with an osteotome. More particularly, the method illustratively includes of the following steps:
1. Using an instrument, illustratively an osteotome, to select the correct staple size and proper placement of the spinal implant. The correct size is selected and properly placed then checked, illustratively through fluoroscopy.
2. Taping (or using another driving source) the osteotome into the bone to form cuts and/or holes (incisions). Blades and awls on the instrument illustratively allow for simultaneous cuts or incisions for the staple legs and holes for the screws, as applicable;
3. Inserting the spinal implant in the bone using mechanical energy (impact force, ultrasonic or sonic energy, reciprocating motion, or other);
4. Tightening screws or fasteners using a screwdriver while the implant remains within the grasp of the insertion instrument. The pre-cut holes from step 2 above allow the screws to gain bone purchase (or engagement) immediately reducing the potential of dislodging the spinal implant during tightening of the screws; and
5. Releasing the spinal implant and removing the instruments.

A further illustrative method includes the additional step of using a centering portion on the bridge of the spinal implant to center and stabilize the implant in the insertion instrument, prior to inserting the spinal implant in the bone.

Additional features and advantages of the present invention will become apparent to those skilled in the art upon consideration of the following detailed description of the illustrative embodiment exemplifying the best mode of carrying out the invention as presently perceived.

### BRIEF DESCRIPTION OF THE DRAWINGS

The detailed description of the drawings particularly refers to the accompanying figures in which:

FIG. 1 is a front view an illustrative embodiment spinal implant or staple, with screws pre-assembled;

FIG. 2 is a front view of the staple and screws in the spine following placement and tightening of the screws;

FIG. 3A is an isometric view of a further illustrative embodiment staple, showing a centering portion extending upwardly from the bridge member;

FIG. 3B is a side elevational view of the staple of FIG. 3A, with screws supported therein;

FIG. 4A is an isometric view of another illustrative embodiment staple, showing centering portions extending laterally outwardly from the bridge member;

FIG. 4B is a side elevational view of the staple of FIG. 4A;

FIG. 5 is an isometric view of an illustrative embodiment osteotome;

FIG. 6 is an isometric view of the end effector of the osteotome of FIG. 5;

FIG. 7 is an exploded view of the end effector of the osteotome of FIG. 5;

FIG. 8 is an isometric view of an illustrative articulated osteotome;

FIG. 9 is an exploded view of the articulated osteotome of FIG. 8;

FIG. 10 is an isometric view of an illustrative powered (ultrasonic or reciprocating motion) insertion instrument;

FIG. 11 is an isometric view of an illustrative embodiment insertion instrument;

FIG. 12 is an isometric view of the end effector of the insertion instrument of FIG. 11;

FIG. 13 is an exploded view of the end effector of FIG. 12;

FIG. 14 is an exploded view of the handle of the insertion instrument of FIG. 11;

FIG. 15 is an isometric view of the insertion instrument of FIG. 11 with a spinal implant device or staple installed and ready for placement;

FIG. 16 is an isometric view of a further illustrative embodiment insertion instrument;

FIG. 17 is an isometric view of the end effector of the insertion instrument of FIG. 16, ready for engagement with the screws and staple;

FIG. 18 is a front view of the pre-assembled staple and screws attached to the insertion instrument of FIG. 16;

FIG. 19 is an isometric view of a further illustrative embodiment osteotome instrument;

FIG. 20 is an isometric view of an end effector of the osteotome instrument of FIG. 19;

FIG. 21 is an isometric view of another illustrative embodiment insertion instrument;

FIG. 22 is a cross-sectional view taken along line 22-22 of FIG. 21;

FIG. 23 is an isometric view of the end effector of the insertion instrument of FIG. 21, with the jaws in an open position;

FIG. 24 is an isometric view of the end effector similar to FIG. 23, with a staple, including fasteners supported therein, positioned adjacent the open jaws;

FIG. 25 is an isometric view of the end effector similar to FIG. 24, with the jaws in a closed position grasping the staple;

FIG. 26 is an exploded perspective view of the insertion instrument of FIG. 21;

FIG. 27 is a partial cross-sectional view taken along line 27-27 of FIG. 22, with the jaws in an open position, and the lower handle member and the outer shaft in an up or retracted position;

FIG. 28 is a partial cross-sectional view similar to FIG. 27, with the jaws in a closed position grasping the staple, and the lower handle member and the outer shaft in a down or extended position;

FIG. 29 is an isometric view of an illustrative embodiment screwdriver;

FIG. 30 is an isometric view of the hex ball end of the screwdriver of FIG. 29; and

FIG. 31 is an isometric view of the screwdriver of FIG. 29 coupled to a screw pre-assembled in the staple.

### DETAILED DESCRIPTION OF THE DRAWINGS

### Illustrative Spinal Implants

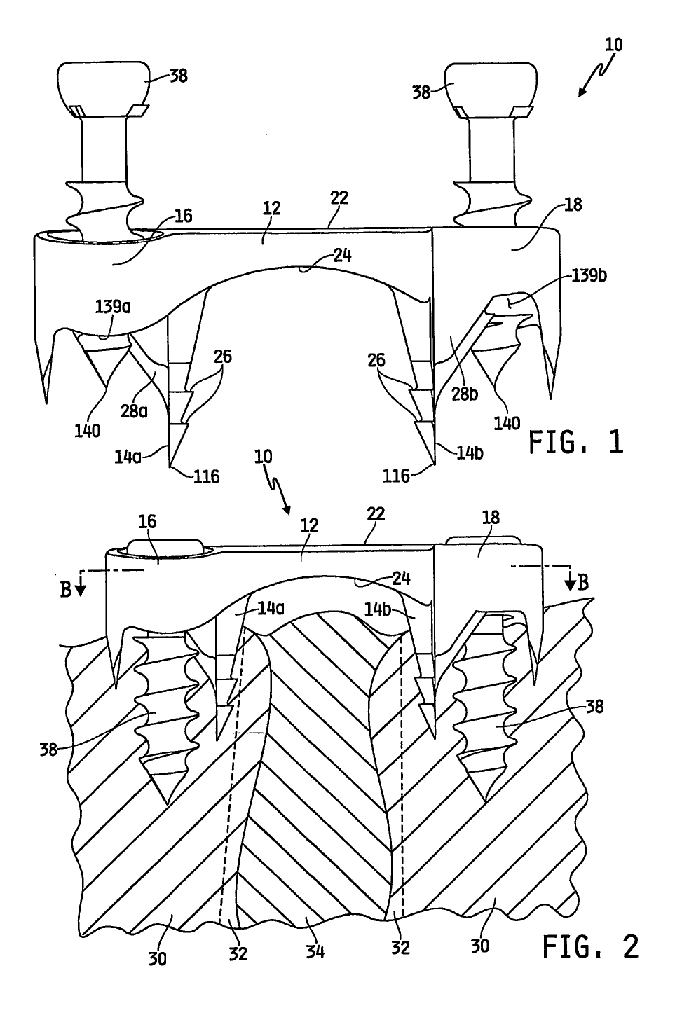

The instrumentation of the present disclosure may find use with a wide variety of orthopedic implants, including those associated with spinal correction systems. For example, the implant device could be a cervical plate or any implant that requires the use or placement of anchors or fasteners. An illustrative spinal correction system including a spinal implant or staple 10 is shown in FIGS. 1 and 2 as including a bridge member 12, a pair of spaced apart legs 14, a left fastener retaining portion 16, and a right fastener retaining portion 18. Although reference may be made throughout this description to terms implying direction, such as left, right, front, back, upper, and lower, unless otherwise noted, these terms are used only for convenience and should not be read as limiting the staple 10 or associated instrumentation to any particular orientation.

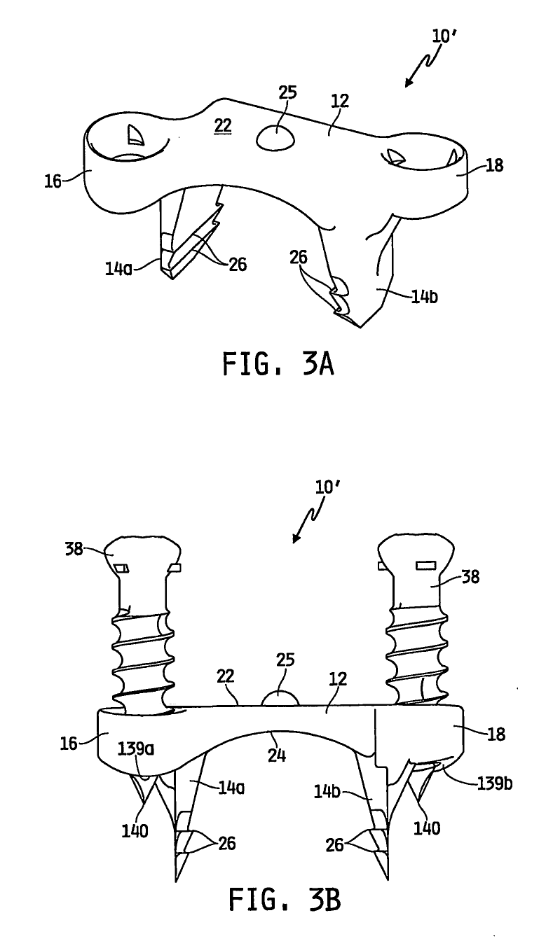

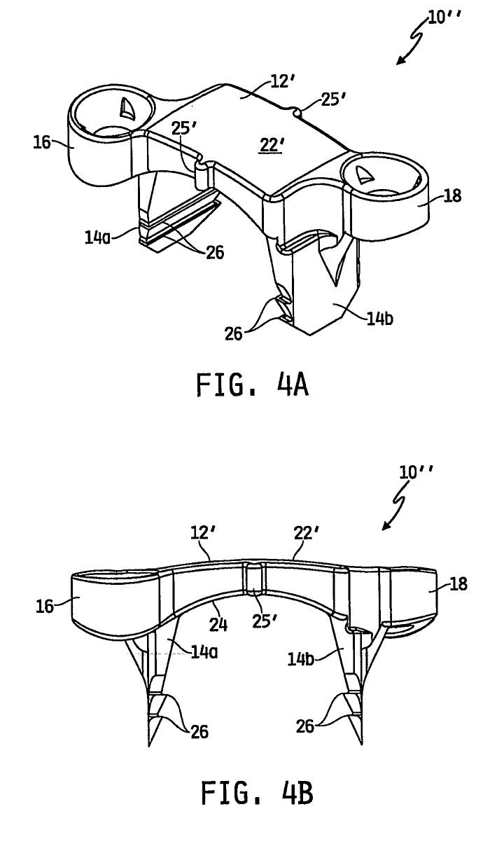

The bridge member 12 couples the left fastener retaining portion 16 to the right fastener retaining portion 18. The lower surface 24 of the bridge member 12 is illustratively concave in a direction from a left end to a right end, and from a front side to a back side. As shown in FIGS. 3 and 4, in a further illustrative embodiment spinal staple 10′, the upper surface 22 of the bridge member 12 may support a centering portion 25, illustratively an upwardly extending spherical protrusion or dimple. Alternative embodiment centering portions 25′ may also be provided, such as the arcuate protrusions extending laterally outwardly from the bridge member 12′ of illustrative spinal staple 10″, as shown in FIGS. 4A and 4B. As illustrated in FIG. 4B, the upper surface 22′ of the bridge member 12′ is illustratively convex in a direction from a left end to a right end. The convex upper surface 22′ provides additional thickness proximate the center of the bridge member 12′.

Left and right legs 14a and 14b extend downwardly from the lower surface 24 proximate the left and right ends of the bridge member 12. Barbs 26 illustratively project outwardly from the legs 14. An anti-rotation or stabilization member or plate 28 may illustratively be located outboard of, and perpendicular to, each leg 14. More particularly, a left anti-rotation member 28a extends between the left fastener retaining portion 16 and the left leg 14a, and a right anti-rotation member 28b extends between the right fastener retaining portion 18 and the right leg 14b. The anti-rotation members 28 are configured to reduce relative motion between adjacent vertebrae 30, while also preventing relative rotation of the fastener retaining portions 16 and 18.

With further reference to FIG. 2, staples 10 illustratively may be inserted into the vertebrae 30 of an animal having an immature or growing spine exhibiting scoliosis or other spinal deformity. The legs 14 are configured such that the staple 10 will bridge longitudinally or lengthwise aligned, adjoining vertebrae 30 having confronting endplate growth centers 32, and an intervening disc 34 therebetween. The staples 10 are illustratively driven into the bone of adjoining vertebrae 30 on the convex side of the curved spine.

Once a staple 10 is in place, fasteners 38, such as screws including threaded portions, may be inserted into the vertebrae 60 to further secure the fastener retaining portions 16 and 18 to the spine. Illustratively, the fastener retaining portions 16 and 18 may include threads to engage the threads of the fasteners 38. Additional details of illustrative embodiment staples 10 are provided in U.S. patent application Ser. No. 11/126,782, filed May 11, 2005, the disclosure of which has been incorporated by reference herein.

### First Set of Illustrative Instruments

Illustrative Osteotomes (Chisel Tools)

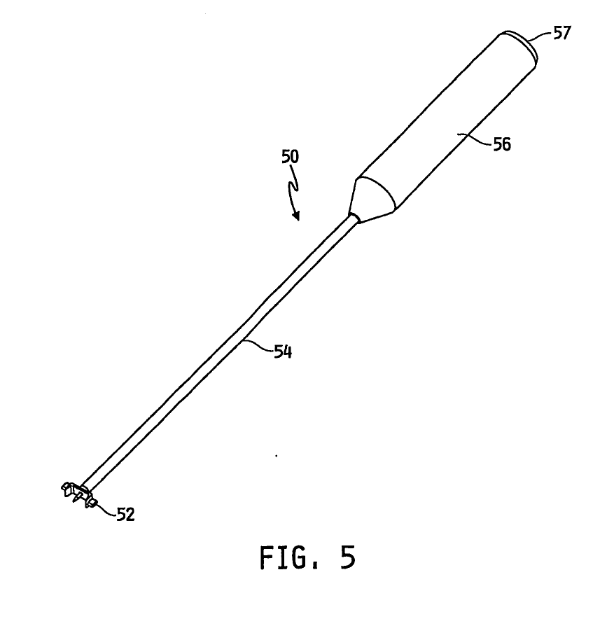

FIG. 5 shows an illustrative embodiment osteotome 50, or chisel tool, with an end effector 52, a shaft 54, and a handle 56. More particularly, the shaft 54 couples the end effector 52 to the handle 56. An upper end 57 of the handle 56 is illustratively used for applying force, such as impact from a mallet (not shown). It should be appreciated that energy may be applied in other manners, as detailed herein The shaft 54 illustratively has a length of at least approximately 20 centimeters in order to facilitate endoscopic access.

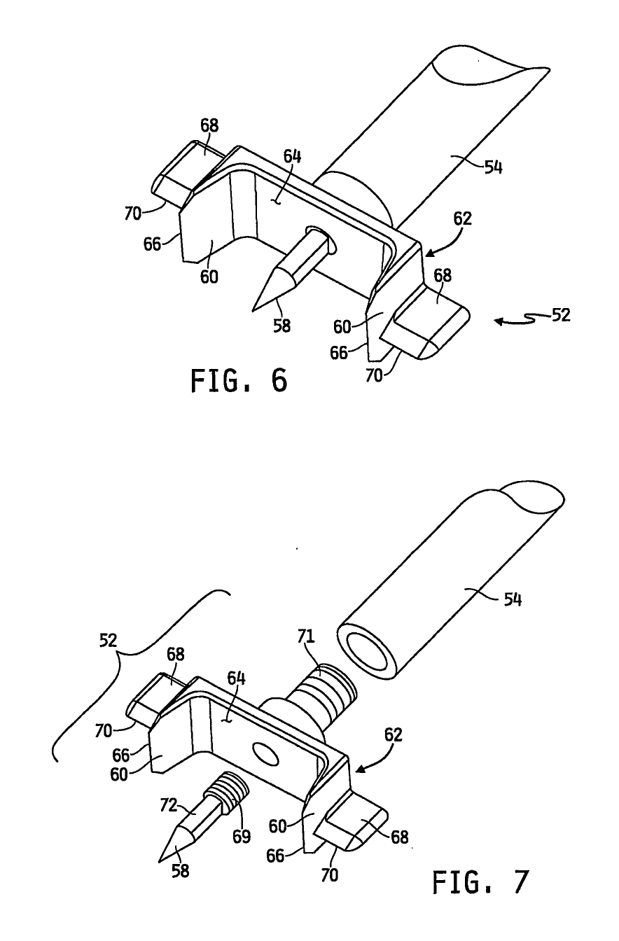

Additional details of the illustrative end effector 52 are shown in FIGS. 6 and 7. A sharp alignment needle 58 is used to penetrate the soft tissue of the intervening disc 34 prior to contact by a pair of cutting or chisel blades 60. The blades 60 are coupled to an osteotome body or yoke 62 and are configured to cut through overlying tissue, as well as into the compact or cortical bone (outer layer) of the vertebrae 30 for subsequent placement of the legs 14 of the spinal staple 10. The alignment needle 58 is relatively small in diameter and short to reduce the depth of penetration into a structure (e.g., disc 34) prior to and following cutting. Illustratively, the needle 58 has a length not exceeding approximately 30 millimeters as measured from a lower stop surface 64, and an outer diameter not exceeding approximately 2 millimeters. The maximum length of the needle 58 is configured to not exceed approximately one half the respective vertebral diameter into which the needle 58 is to be inserted. Illustratively, the alignment needle 58 protrudes immediately beyond sharp or cutting edges 66 of the blades 60.

In operation, the surgeon assures the alignment needle 58 is centered in the vertebral disc 34 by observing the blades 60 to see where incisions will be made by the sharp edges 66. Alternatively, imaging such as radiography or fluoroscopy may be used to plan the placement of the staple 10 to assure centering in the disc 34 and accurate placement in relation to the vertebral growth plates. Supplementary or secondary anti-rotational plate blades 68 with sharp or cutting edges 70 are used to cut vertebral bone for other features, and are illustratively positioned 90-degrees from the blades 60 for subsequent placement of anti-rotation plates 28 of the spinal implant or staple 10. As shown, the supplementary blades 68 are coupled to the yoke 62 and extend outwardly from the cutting blades 60. Following proper placement, the sharpened edges 66 of the blades 60 are placed on the tissue or bone and a mechanical impacting device such as a mallet is used to apply energy or strike the end 57 of the handle 56 driving the blades 60 into the vertical bone. The supplementary blades 68 cut the bone once the blades 60 have penetrated the tissue and bone. Timing and depth of the blade cut in relation to the cutting blades 60 is controlled by relative longitudinal position. In this embodiment, the supplementary blades 68 enter the bone after the sharp edge 66 of the blades 60 have penetrated the bone and therefore penetrate the bone to a shallow depth. The depth of cut is controlled by the length of the blade 60 from the stop surface 64.

The end effector 52 is illustratively assembled as shown in FIG. 7. The alignment needle 58 illustratively includes threads 69 for attachment to the osteotome yoke 62 which, in turn, includes threads 71 for attachment to the shaft 54. Flats 72 on the needle 58 allows for griping with a tool such as a wrench (not shown) for assembly or replacement of the needle 58. Likewise if the osteotome yoke 62 should become dull, it may be replaced. Once the bone is precut, the osteotome 50 may be withdrawn. Withdrawal may be facilitated by angular or rotational motion applied at the handle 56.

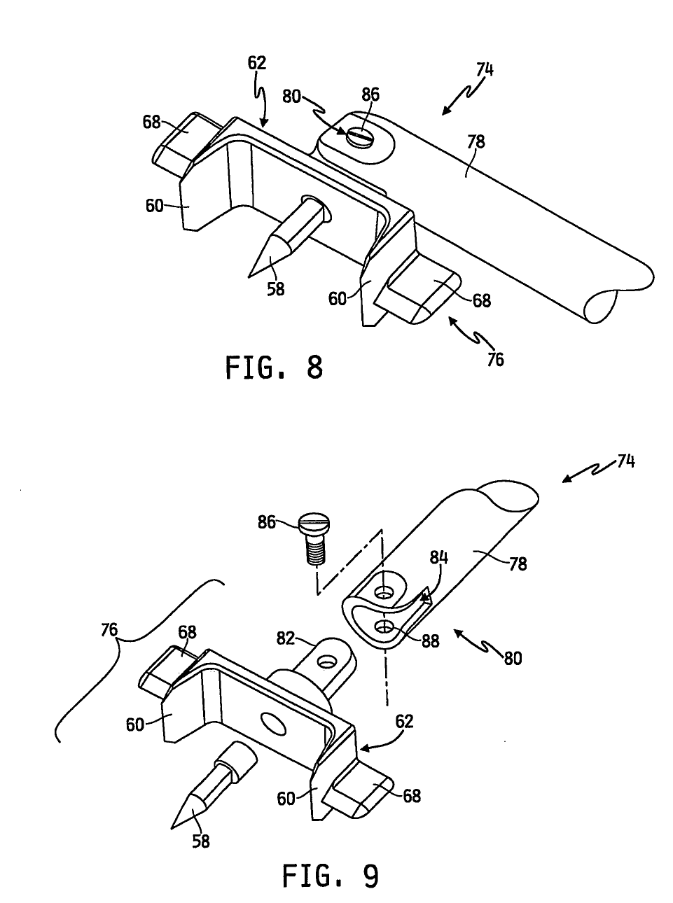

FIGS. 8 and 9 show a further illustrative embodiment osteotome 74, or chisel tool with end effector 76 and shaft 78 such that the end effector 76 may be articulated 90-degrees to the shaft 78. The shaft 78 illustratively has a length of at least approximately 20 centimeters in order to facilitate endoscopic access through a port and is articulated by a hinge joint 80. The joint 80 includes a clevis 82 which is a part of the end effector 76, and that fits into a half slot 84, and is pinned in the slot 84 by a pivot bolt 86. The pivot bolt 86 is held in place by tightening within a threaded hole 88. Detents (not shown) may be added to assure the end effector 76 remains in the articulated position or the ready to cut position. The articulation may be operated manually, or may have a remote operation if desired.

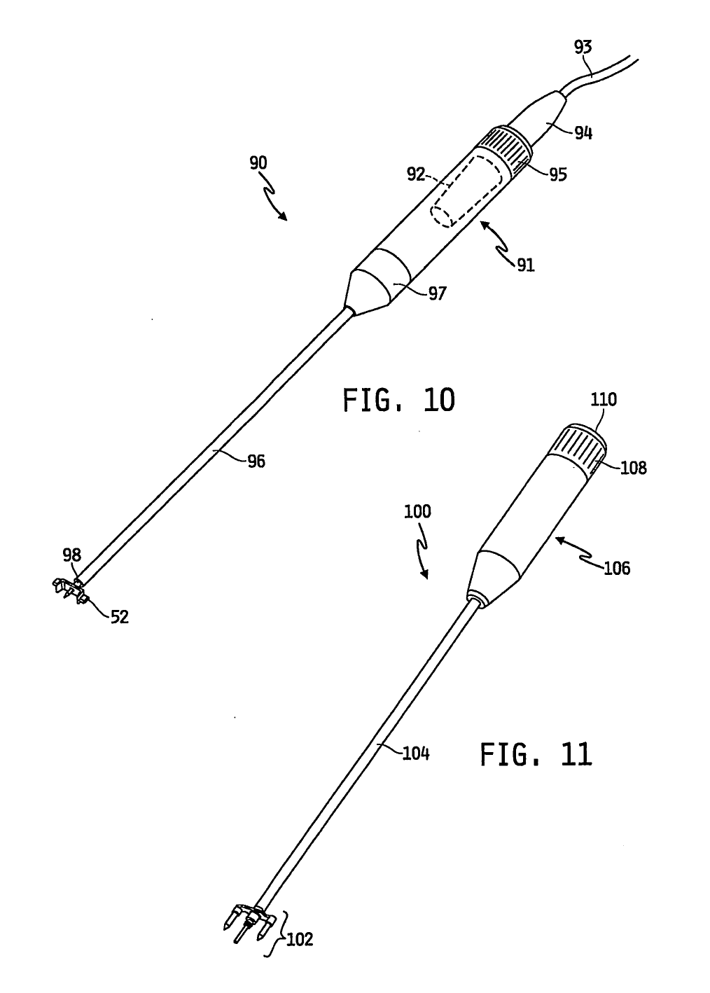

FIG. 10 shows an isometric view of an alternative embodiment ultrasonic or powered osteotome 90. Illustratively, osteotome 90 includes a handle 91 including an actuator or motor 92 connected to a generator/controller (not shown) via a power cable 93 having a strain relief 94. The handle 91 has a release-operating knob 95 and attaches to an outer shaft or sleeve 96 via a thread cap 97. An inner shaft 98 extends from motor 92 located inside the handle 91 to the end effector 52. The inner shaft 98 transmits ultrasonic energy of reciprocating motion from the motion in the handle 91 to the end effector 52 when the motor 92 is energized by the generator/controller via the power cable 93. More particularly, reciprocating actuator or motor 92 located inside the handle 91 is connected to the inner shaft 98 for transmitting reciprocating energy generated by the motor to the end effector 52. The sleeve 96 illustratively has a length of at least approximately 20 centimeters in order to facilitate endoscopic access and covers the inner shaft 98. The inner shaft 98 can be moved longitudinally with respect to the sleeve 96 using the release knob 95 that allows detachment of the end effector 52 following placement. The thread cap 97 attaches the outer shaft 96 to the handle 91 via a set of threads (or other means of attachment).

Illustrative Insertion Instruments (Placement Tools)

An illustrative embodiment insertion instrument 100, or placement tool, is shown in FIG. 11 for grasping and inserting a spinal implant or staple 10, illustratively following use of the osteotome 50, 90 in the manner detailed above. The insertion instrument 100 includes an end effector 102, a sleeve or outer shaft 104 and a handle 106. The outer shaft 104 couples the end effector 102 to the handle 106. The handle 106 illustratively includes a rotatable release-operating knob 108. The outer shaft 104 illustratively has a length of at least approximately 20 centimeters in order to facilitate endoscopic access. The handle end 110 is useful for impacting, although other forms of energy may be used for insertion of the implant or staple 10.

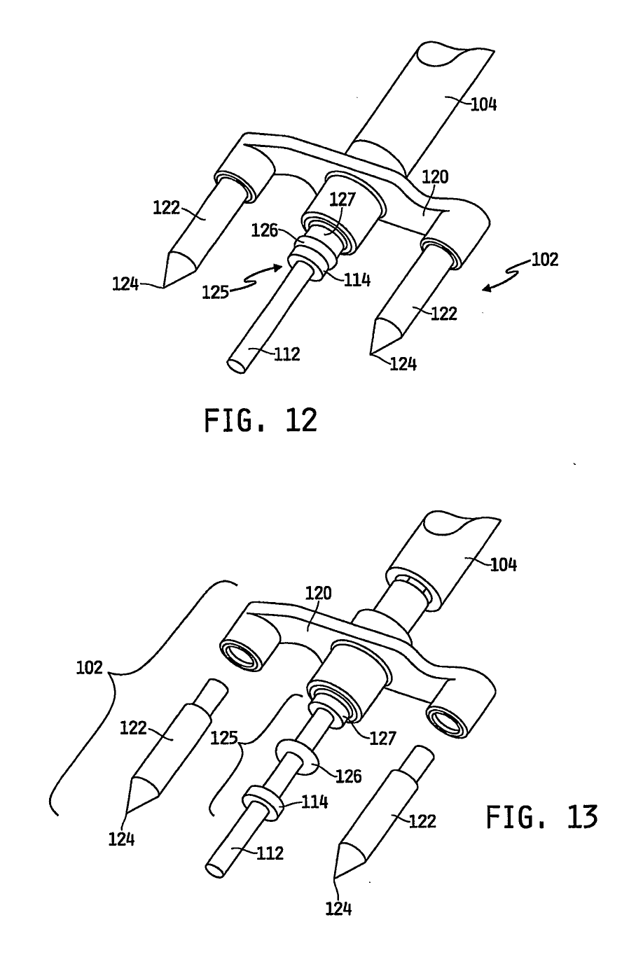

FIG. 12 shows the end effector 102 and an inner shaft, blunt wire, or pin 112 supported within the outer shaft 104. Illustratively, inner shaft 112 has dimensions similar to those of the needle 58 detailed herein, wherein the length of inner shaft 112, as measured from a compressor ring 114, illustratively does not exceed approximately 30 millimeters. As shown in FIG. 15, the inner shaft 112 illustratively does not extend significantly beyond the blade tips 116 of the staple 10. FIG. 13 is an exploded view of the end effector 102. A yoke or body 120 of the end effector 102 is attached to the outer shaft 104. One or more awls 122 with sharp points 124 are assembled to the yoke 120 and are configured to cut bone for subsequent placement of fasteners 38.

A gripping or locking mechanism 125 is also operably coupled to the end effector 102 and is configured to releasably retain the spinal staple 10. More particularly, the inner shaft 112 supports a wide washer-like compressor ring 114 that is moved longitudinally by rotating the release-operating knob 108 on the handle 106. A retaining ring 126 is also supported by the inner shaft 112 and is illustratively made of a resilient material such as elastomeric rubber. As the compressor ring 114 compresses the retaining ring 126 against a stop ring 127 of the yoke 120, the ring 126 will expand outwardly increasing in diameter. This increased diameter of the retaining ring 126 is used to grip the spinal implant or staple 10 as shown in FIG. 15. This griping or locking mechanism 125 is not limiting, in that other mechanisms may be used for releasably gripping the implant or staple 10 such that it is easily released with slight motion of the handle 106.

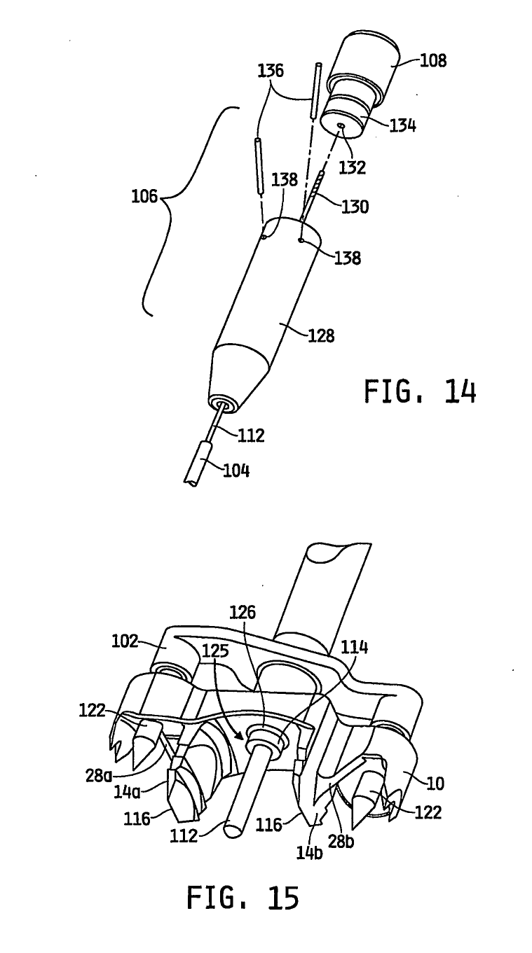

FIG. 14 shows an exploded view of the illustrative handle 106. The purpose for the handle 106 is to provide a mechanical means to compress and decompress the retaining ring 126 for selectively griping and releasing the spinal implant device or staple 10. The handle 106 illustratively includes knob 108, and a handle base 128 that assembles to the outer shaft 104. Inner shaft 112 includes threads 130 such that it may be screwed into a threaded hole 132 in the knob 108. The knob 108 has a retaining groove 134. The handle 106 is assembled by threading the inner shaft 112 in the threaded hole 132, placing the knob 108 in the end of the handle base 128, and then placing pins 136 in retaining holes 138 such that the pins 136 loosely engage the retaining groove 134. The outer shaft 104 is attached to the handle base 128 by conventional means such as threads, adhesive, or a press fit. This assembly allows the knob 108 to be rotated thereby moving the inner shaft 112 longitudinally relative to the handle base 128 and outer shaft 104. Illustratively, stops (not shown) may be provided to define the range of motion for operation of the instrument 100.

FIG. 15 shows the end effector 102 with the spinal implant or staple 10 attached. The blunt wire or inner shaft 112 of the end effector 102 is illustratively used to find the datum hole previously made by the osteotome needle 58 in the tissue or disc 34 and is optionally marked via a dye for easy location. More particularly, the datum hole is used as a datum to assure the insertion instrument 100 can find the exact location of the cuts made by the osteotome 50 for receipt of the legs 14 and anti-rotation plates 28 of the spinal staple 10. The retaining ring 126, when compressed by the compressor ring 114, expands to grip the spinal implant or staple 10 for manipulation and placement. The resilience of the retaining ring 126 allows some relative movement of the spinal implant or staple 10 to assure the precut bone can guide the spinal staple 10 in the preplanned location.

After the spinal staple 10 location is established via the spinal datum hole, the implant instrument can be mechanically impacted to force the staple 10 and the awls 122 into the vertebrae 30, or optionally some other form of energy can be used for insertion. The knob 108 can then be rotated allowing the inner shaft 112 to move such that the compressor ring 114 decompresses the retaining ring 126 and releases the spinal implant device or staple 10. Should the insertion instrument 100 become wedged or otherwise stuck in the spinal staple 10 as a result of impact or application of other energy sources, applying some relative motion at the handle base 128 will allow easy removal by breaking it loose. The insertion instrument 100 may then be removed without dislodging the spinal staple 10. The retaining ring 126 allows motion due to its resilience without dislodging the staple 10. Once the spinal staple 10 is released and the insertion instrument 100 removed, screws 38 may be inserted through fastener retaining portions 16 and 18 and rotated into threaded engagement with the vertebrae 30.

The illustrative osteotomes and insertion instruments help avoid the risk of damaging or cracking by precutting tissue and bone prior to placement by pre-cutting the bone and overlying tissue. This allows the surgeon to pre-plan the placement of the spinal implant device or staple 10 and fasteners or screws 38. This pre-cutting and the resilience or flexibility built in the insertion instrument allows then the spinal staple 10 and fasteners 38 to find the preplanned insertion route when placed. Accurate placement and pre-cutting reduces the risk that the bone will be split, or cracked and assure the staple legs 14 and fasteners 38 will not disrupt the vertebral growth plates. The instruments also allow installation through an endoscopic port.

Placing and marking a hole during pre-cutting and then finding the same hole with the blunt wire or inner shaft 112 assists in accurate placement and preventing excess stress from deforming the staple 10, thereby reducing the potential for penetrating the disc 34 or growth plate of the vertebrae 30 with the staple legs 14. This method further allows endoscopic placement of the spinal staple 10 and fasteners 38. The method also requires fewer steps and less time than placing a guide wire in the disc 34 and leaving it in place while the spinal staple 10 is placed. It reduces the potential that the guide wire could be inadvertently pushed through the disc striking vital organs such as the spinal cord. It provides accurate precutting and placement of the spinal staple 10 to assist in avoiding the potential for cracking or splitting of the bone. The method further allows movement of the instruments to permit removal without dislodging the spinal staple 10 after placement.

Powered insertion using ultrasonic or sonic energy, vibration, or low frequency reciprocating allows ease of cutting if applied to the osteotome. Pre-cutting using this method limits the risk of splitting while inserting an implant. The powered cutting methods limit the risk of splitting while inserting an implant. Powered cutting illustratively converts the bone into a fine or smoke like powder that can be suctioned away quickly.

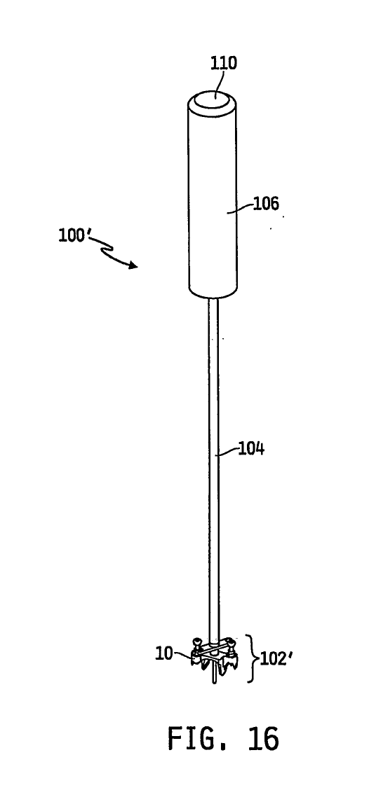

Another illustrative embodiment of the insertion instrument 100′ is shown in FIG. 16 with an end effector 102′, an outer shaft 104, and a handle 106. Again, the shaft 104 illustratively has a length of at least approximately 20 centimeters in order to permit endoscopic access. The handle end 110 is useful for impacting, although other forms of energy may be use for insertion of the spinal staple 10.

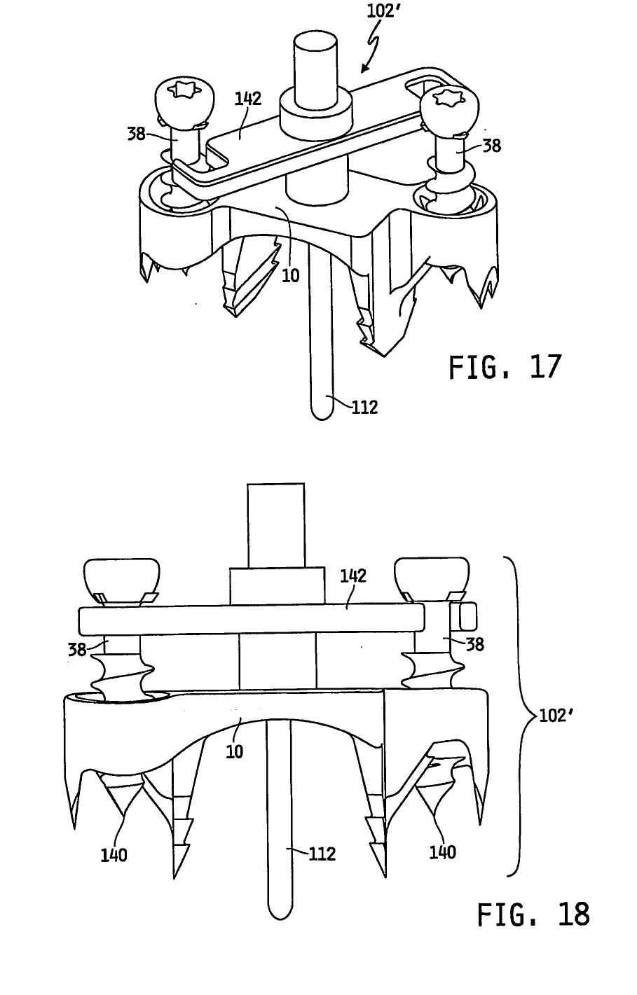

FIGS. 1 and 17 show the staple 10 pre-assembled with fasteners or screws 38 such that the points 140 of the screws 38 are protruding a short distance beyond the distal or lower surfaces 139a and 139b of fastener retaining portions 16 and 18 of staple 10. Illustratively, the screws 38 may be threadably received within the spinal staple 10. FIG. 17 shows the end effector 102′ with the inner shaft 112 inserted in the staple 10, and a yoke 142 ready to be rotated to engage or hold the screws 38 thus engaging or holding the staple 10. FIG. 18 shows the end effector 102′ engaged or holding the screws 38 which are assembled in staple 10. The inner shaft 112 of the end effector 102′ is illustratively used to find the datum hole previously made by the osteotome in the tissue or disc and is optionally marked via a dye for easy location. As noted above, the screws or fasteners 38 are pre-threaded in the staple 10 to where the points 140 of the screws 38 are only slightly protruding below the lower surfaces 139a and 139b of the staple 10. The yoke 142 selectively engages the screws 38 for attachment of the instrument 100′ to the screws 38 and staple 10. Attachment and detachment of the staple 10 and screws 38 can be achieved by rotating the insertion instrument 100′, including outer shaft 104 and yoke 142 about the centerline of the inner shaft or pin 112 in relation to the staple and screws 38 that were pre-assembled. This simple attachment and detachment method reduces the potential of dislodging the staple 10 and reduces the number of steps required for placement.

Once the staple 10 and screws 38 are placed, the yoke 142 is rotated to release the screws 38. Each screw 38 is subsequently tightened. FIG. 2 shows the position of the screws 38 after tightening in the bone or vertebra 30 with the staple 10 bridging the disc 34.

The illustrative embodiment of FIGS. 16-18 reduces the number of steps by pre-assembling the staple 10 and fasteners 38 prior to placement. The method eliminates the potential for dropping fasteners 38 and the time required to place each screw 38. The engagement and disengaging method and mechanism is simple and requires fewer parts. The method reduces the number of steps required for surgery therefore reducing the total surgical procedure time. Reduced time under anesthesia reduces patient risk and operating room costs.

### Second Set of Illustrative Instruments

Illustrative Osteotome (Chisel Tool)

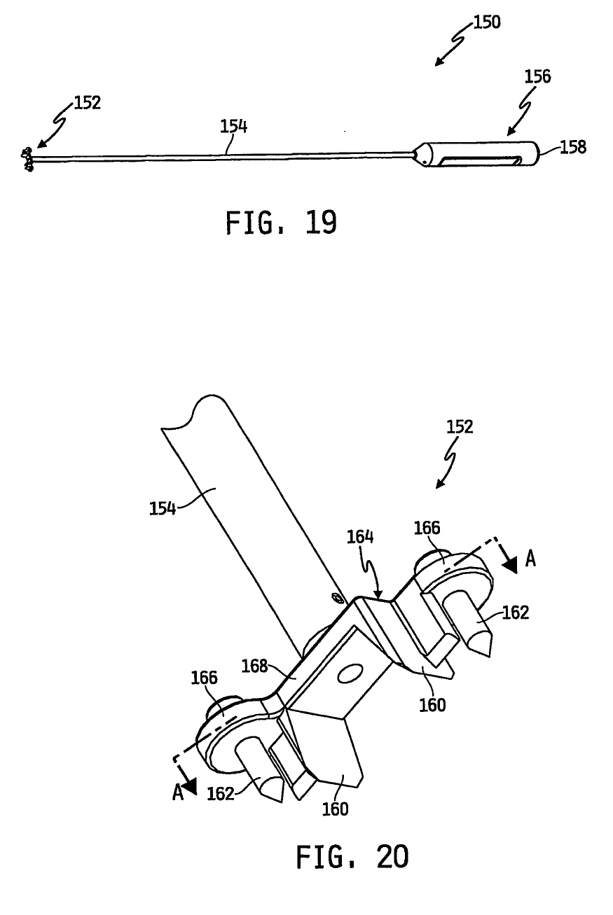

FIG. 19 shows a further illustrative embodiment osteotome 150 configured to pre-cut the overlying tissue (if any) and the vertebral bone. The osteotome 150 includes an end effector 152, a shaft 154, and a handle 156. The end 158 of the handle 156 is useful for impacting with a mallet to drive the osteotome end effector 152 through tissue and bone. A series of osteotomes 150 may be provided, wherein each osteotome 150 is equivalent in size to a corresponding one (or more) of a plurality of spinal correction implants or staples 10′.

FIG. 20 shows the end effector 152 of osteotome 150 with sharp chisel blades 160 and awls 162 for cutting incisions or holes for subsequent placement of the spinal staple 10′. The osteotome 150 is illustratively used to both gauge the correct size of staple 10′ and to pre-cut the bone. The osteotome 150 includes an osteotome body, gauge member, or yoke 164 having at least one gauge flange 166 and a gauge back plate 168, which are used for gauging the size of staple 10 that is suitable for installation. The gauge flanges 166 and gauge back plate 168 have the same peripheral dimensions as the spinal correction implant or staple 10′, thereby facilitating location and fit determination by a surgeon. Moreover, the blades 160 extending downwardly from the gauge back plate 168 are spaced apart to facilitate positioning by the surgeon on adjoining vertebrae 30 while providing clearance for the intervening disc 34 and respective endplate growth centers 32 (FIG. 2).

More particularly, the gauge member 164 has a planar profile, taken along plane “A” of FIG. 20, with peripheral dimensions substantially identical to peripheral dimensions of the anterior-posterior planar profile of the spinal staple 10′, taken along plane “B” of FIG. 2. The end effector 152 is illustratively made of an optically or radio opaque material to allow visualization using fluoroscopy. When impacted on the handle end 158, the chisel blades 160 and awls 162 simultaneously make incisions in the overlying tissue and bone for subsequent receipt of the legs 14 and the screws 38, respectively, of spinal staple 10′. The incisions mark the selected location and allow the staple 10 to drop in for proper placement of the staple 10′. As noted above, the end effector 152 may be viewed by the surgeon, illustratively through fluoroscopy, to ensure proper positioning of the blades 160, and hence subsequent positioning of the legs 14 of the staple 10′ within adjoining vertebrae 30 while providing clearance for the intervening disc 34 and endplate growth centers 32. The sharp chisel blades 160 and awls 162 also reduce the risk of fracturing the bone when the staple 10′ is inserted. Cutting the bone in advance of placing the staple 10′ also allows for the surgeon to inspect the bone prior to insertion for reducing the rare potential of a fracture.

Illustratively, a surgical kit may include a plurality of spinal implants or staples 10′, and a plurality of osteotomes 150 wherein the gauge member 164 of each osteotome has a planar profile with peripheral dimensions substantially identical to peripheral dimensions of the anterior-posterior profile of at least one of the spinal implants 10′. Each surgical kit may also include additional tools, such as at least one insertion instrument 200 and/or screwdriver 238.

Illustrative Insertion Instrument (Placement Tool)

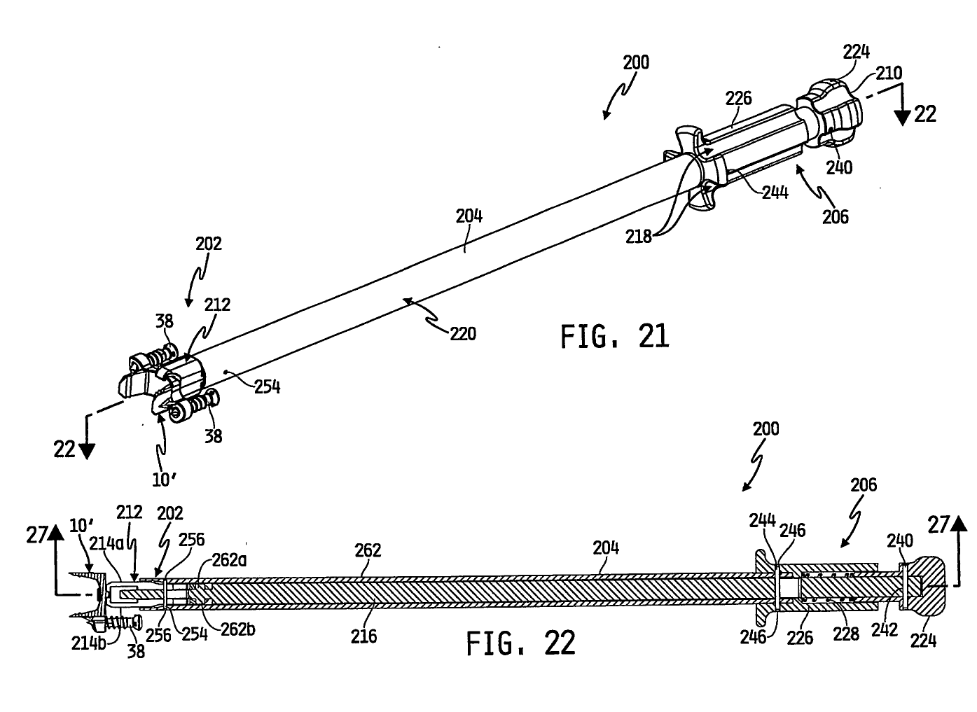

FIG. 21 shows a further illustrative embodiment insertion instrument 200 which is used for grasping and inserting a spinal implant or staple 10′, illustratively following use of the osteotome 150 in the manner detailed above. The insertion instrument 200 has an end effector 202, a sleeve or outer shaft 204, and a handle 206. The insertion instrument shaft 204 illustratively has a length of at least approximately 20 centimeters in order to permit endoscopic access. The handle end 210 is useful for impacting with a mallet (not shown) to drive or insert the spinal staple 10′ into tissue and bone. As detailed above, the implant device could also be a cervical plate or any orthopedic implant.

The end effector 202 illustratively includes a releasable gripping mechanism 212 operably coupled to the handle 206 through an inner shaft or inner shaft 216 received within the outer shaft 204. The releasable gripping mechanism 212 illustratively includes pivotable jaws 214a and 214b that are configured to grasp the staple 10′ firmly. The handle 206 has a plurality of channels 218 to allow a screwdriver or other instruments to be placed next to the shaft 204 directly over or in line with the centerline of the screws 38.

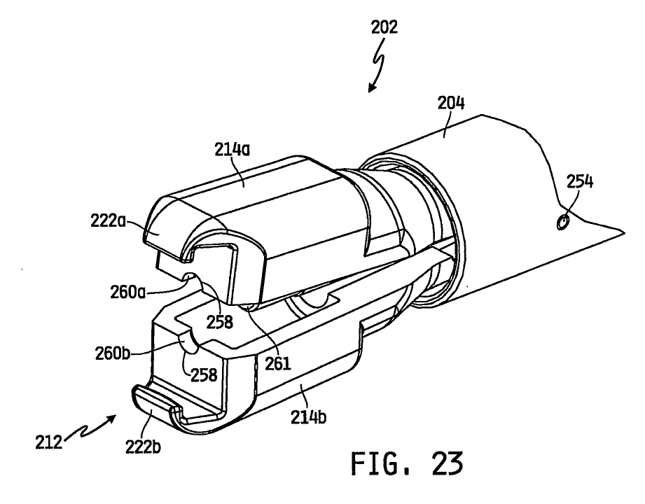

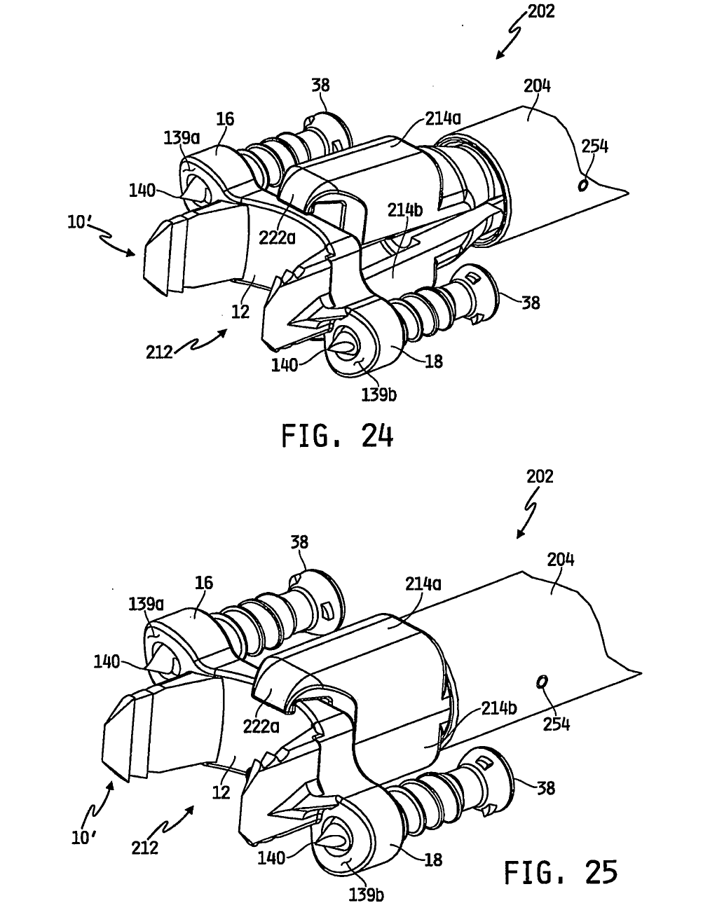

FIGS. 23 and 24 show the end effector 202 with the jaws 214a and 214b open and ready to grasp the spinal correction implant or staple 10′. Fasteners or screws 38 are pre-assembled in the fastener retaining portions 16 and 18 of the staple 10′ such that the points 140 of the screws 38 protrude a short distance beyond the distal or lower surfaces 139a and 139b of the fastener retaining portions 16 and 18. A close fit between the internal threads of the fastener retaining portions 16 and 18 and the external threads of the screws 38 assure the screws 38 remain in place during handling and placement. In certain embodiments, an interference fit may be provided between the fastener retaining portions 16 and 18 and the screws 38.

FIG. 25 shows the end effector 202 grasping the spinal correction implant or staple 10′. As detailed herein, the jaws 214a and 214b are moved toward each other and held in a closed position by the wall 220 of the shaft 204. The jaws 214a and 214b each have a jaw tab 222a and 222b that wraps around the staple 10′ to firmly grasp it.

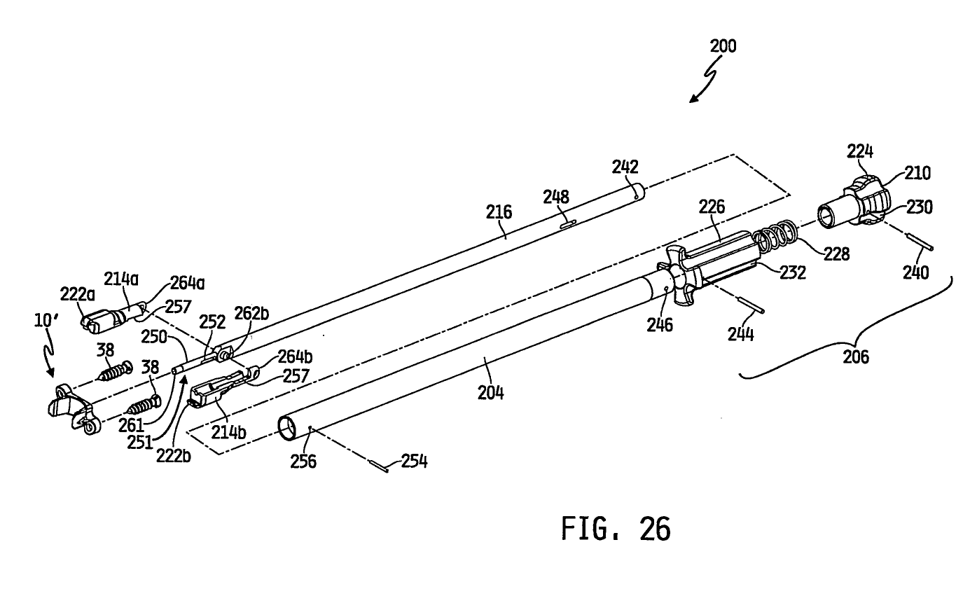

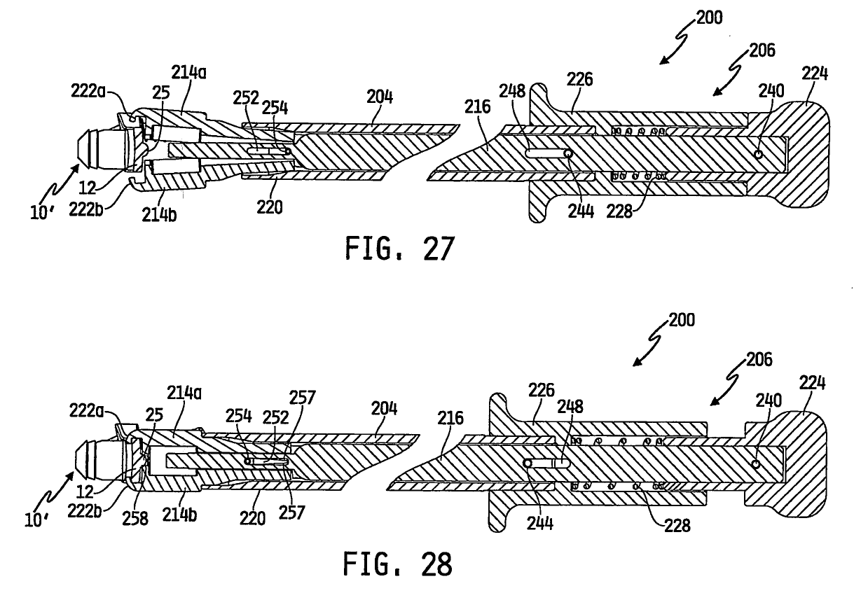

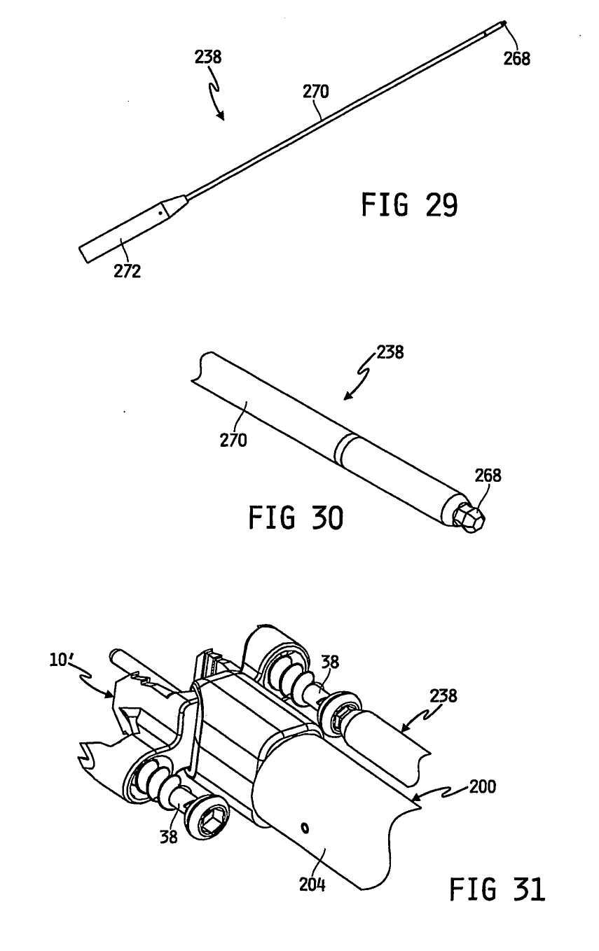

FIGS. 21, 22, 25 and 28 are various views of the instrument 200 with the jaws 214 closed around the spinal staple 10′. FIGS. 23 and 27 show the jaws 214 are open. FIG. 26 is an exploded view of the insertion instrument 200 together with the staple 10′ and screws 38. The handle 206 is an assembly of an upper knob 224, a lower handle member 226, and a coil spring 228 configured to bias the lower handle member 226 away from the upper knob 224. The handle 200 includes channels 218 to permit placement of instruments next to the shaft 204. The channels 218 are formed by recesses 230 and 232 in both the upper knob 224 and the lower handle member 226 and are in alignment with each other. Two of the channels 218 are configured to align with the screws 38 supported within staple 10′ when positioned in the end effector 202, thereby allowing placement of an instrument such as a screwdriver 238 (FIG. 29) in close proximity to the shaft 204, as shown in FIG. 31.

The upper knob 224 is attached to the inner shaft 216 by an upper pin 240 that is pressed in an upper pin hole 242 in the inner shaft 216. The upper knob 224 as attached to the inner shaft 216 is used to transmit force or impact applied to the handle end 210 to the jaws 214a and 214b to the staple 10′ for driving it into tissue or bone or more specifically into the vertebral body of the spine. The lower handle member 226 is attached to the outer shaft 204 by a lower pin 244, which is received within pin holes 246 formed within the outer shaft 204 and extends through a guide slot 248 formed in the inner shaft 216. As such, the lower handle member 226 and outer shaft 204 are movable in relation to the inner shaft 216. The inner shaft 216 has a portion 250 of smaller diameter at the distal end 251 which also has a guide slot 252. The outer shaft 204 is further guided in motion relative to the inner shaft 216 by a shaft cross pin 254, which is received within pin holes 256 formed within the outer shaft 204 and extends through the guide slot 252 of the inner shaft 216. Movement of the cross pin 254 within the guide slot 252 facilitates relative movement of the outer shaft 204 and the inner shaft 216, while also causing relative movement of the jaws 214a and 214b from a closed to an open position. More particularly, each jaw 214a and 214b includes a ramp or cam surface 257 configured to be engaged by the cross pin 254 as it moves within the slot 252 in a direction away from the distal end 251 of the inner shaft 216. In other words, as the outer shaft 204 is moved away from the distal end 251 of the inner shaft 216, the cross pin 254 moves within the slot 252 and engages the cam surfaces 257 of the arms 214a and 214b, thereby causing the arms 214a and 214b to pivot away from each other.

A centering member 258, such as a centering hole is illustratively defined by the end effector 202 and configured to engage with the staple centering portion 25 (FIG. 3) to center and stabilize the spinal correction implant device or staple 10′ in the jaws 214a and 214b of the insertion instrument 200. The centering member 258 is illustratively defined by a pair of arcuate notches 260a and 260b in the jaws 214a and 214b. In further illustrative embodiments, a center recess 261 may be formed at the distal end 251 of the inner shaft 216 for engagement with the centering portion 25 for centering and stabilizing the staple 10′. It should be appreciated that other centering members may be formed on the end effector 202, such as in side surfaces of the jaws 214 to operably couple with the centering portions 25′ of the illustrative staple 10″ (FIGS. 4A and 4B).

The jaws 214a and 214b are pivotally attached to the inner shaft 216 by pivot bosses 262 that are received within holes 264 in the jaws 214a and 214b such that they can pivot relative to each other. The pivoting action allows the jaws 214a and 214b to open and close to grasp the staple 10′. As shown in FIG. 27, the jaws 214a and 214b are in an open position when the lower handle member 226, and hence the outer shaft 204, are in an up or retracted position. In the manner detailed above, in the open position the cross pin 204 engages cam surfaces 257, thereby causing the jaws 214a and 214b to be pivoted away from each other. As shown in FIG. 28, the jaws 214a and 214b are held in the closed position by the outer shaft 204 when the lower handle member 226, and hence the outer shaft 204, are in a down or extended position. More particularly, the cross pin 204 moves away from the cam surfaces 257, and the side wall 220 of the shaft 204 forces the jaws 214a and 214b together when the outer shaft 204 is moved in a distal direction from the open position of FIG. 27 to the closed position of FIG. 28.

Illustrative Screw Driver

FIG. 29 shows a view of the screw driver 238 with a hex ball end 268 coupled to a screwdriver shaft 270. A handle 272 is also coupled to the shaft 270. The shaft 270 is long enough to be used with the insertion/extraction instrument 200. More particularly, the shaft 270 has a length sufficient to clear the handle 206 of the insertion instrument 200, such that the screw driver 238 and the insertion instrument 200 may be used in tandem. The hex ball end 260 allows access to the screws 38 at various angles.

FIG. 30 shows close-up view of the screwdriver hex ball end 268 on the end of the screwdriver shaft 270. FIG. 31 shows the insertion/extraction instrument shaft 204 and end effector 202 grasping the spinal correction implant or staple 10′ with fasteners or screws 38 in place. The screwdriver shaft 270 is in close proximity to the instrument shaft 204 and is engaged with a screw 38 for tightening. The screwdriver shaft 270 extends up through the handle channels 218 to allow it to be adjacent to the shaft 204.

Although the invention has been described in detail with reference to certain preferred embodiments, variations and modifications exist within the spirit and scope of the invention as described and defined in the following claims.

1. An osteotome configured to prepare a spine for the receipt of a spinal implant, the osteotome comprising:
a handle;
a shaft coupled to the handle; and
an end effector coupled to the shaft and including an alignment needle configured to define a datum and a cutting member configured to precut bone for the subsequent insertion of a spinal implant device.

2. The osteotome of claim 1, wherein the cutting member includes a pair of laterally extending cutting blades positioned on opposite sides of the alignment needle and configured to penetrate bone in preparation for receiving legs of a spinal implant.

3. The osteotome of claim 2, wherein the cutting member further includes a pair of longitudinally extending cutting blades positioned substantially perpendicular to the laterally extending cutting blades and extending in a direction outwardly relative to the alignment needle, the longitudinally extending cutting blades being configured to penetrate bone in preparation for receiving anti-rotation plates of a spinal implant.

4. The osteotome of claim 1, wherein the end effector further includes a stop surface configured to control the depth of cut into bone by the cutting member.

5. The osteotome of claim 1, wherein the end effector includes a yoke, the alignment needle being releasably coupled to the yoke.

6. The osteotome of claim 1, wherein the end effector includes a yoke, the shaft being releasably coupled to the yoke.

7. An osteotome configured to prepare a spine for the receipt of a spinal implant, the osteotome comprising:
a handle;
a shaft coupled to the handle; and
an end effector coupled to the shaft and including a gauge member and a pair of cutting members extending downwardly from the gauge member, the gauge member having a gauge back plate and at least one gauge flange extending outwardly from the gauge back plate, and the cutting members configured to select the proper placement and precut the bone for subsequent receipt of a pair of legs of the spinal implant.

8. The osteotome of claim 7, wherein the end effector further includes a pair of awls configured to precut the bone for subsequent receipt of the pair of fasteners of the spinal implant.

9. The osteotome of claim 7, wherein the gauge member is formed of an optically opaque material for visualization through use of fluoroscopy.

10. The osteotome of claim 7, wherein the gauge member includes a planar profile with peripheral dimensions substantially identical to peripheral dimensions of the anterior-posterior planar profile of the spinal implant.

11. A surgical kit including:
a plurality of spinal implants, each implant having an anterior-posterior planar profile with peripheral dimensions; and
a plurality of osteotomes, each including a gauge member having a planar profile with peripheral dimensions substantially identical to peripheral dimensions of the anterior-posterior planar profile of at least one of the spinal implants.

12. The surgical kit of claim 11, wherein the gauge member of each of the plurality of osteotomes includes a gauge back plate and at least one gauge flange extending outwardly from the gauge back plate.

13. The surgical kit of claim 11, wherein each of the plurality of osteotomes further includes a handle, and a shaft coupled to the handle and the gauge member.

14. The surgical kit of claim 11, wherein each of the plurality of spinal implants further includes a pair of legs configured to engage bone, and each of the plurality of osteotomes further includes a pair of chisel blades configured to precut the bone for subsequent receipt of the pair of legs of the spinal implant.

15. The surgical kit of claim 11, wherein each of the plurality of spinal implants further includes a pair of fasteners configured to engage bone, and each of the plurality of osteotomes further includes a pair of awls configured to precut the bone for subsequent receipt of the pair of fasteners of the spinal implant.

16. The surgical kit of claim 11, wherein the gauge member is formed of an optically opaque material for visualization through use of fluoroscopy.

17. An insertion instrument configured to manipulate a spinal implant, the insertion instrument comprising:
a handle;
a shaft coupled to the handle; and
an end effector operably coupled to the shaft and including a releasable gripping mechanism configured to releasably grip a spinal implant device.

18. The insertion instrument of claim 17, wherein the releasable gripping mechanism includes a retaining ring and a compressor ring operably coupled to the retaining ring.

19. The insertion instrument of claim 18, wherein the compressor ring is configured to move axially for compressing the retaining ring and thereby increase the outer diameter of the retaining ring.

20. The insertion instrument of claim 17, wherein the end effector further includes a yoke and an awl coupled to the yoke for pre-cutting the bone for subsequent insertion of a fastener.

21. The insertion instrument of claim 17, further comprising an actuator operably coupled to the shaft for transmitting energy to the end effector.

22. The insertion instrument of claim 17, wherein the releasable gripping member includes a pair of movable jaws configured to engage opposing sides of the spinal implant.

23. The insertion instrument of claim 22, wherein each of the jaws include a tab that wraps around a bridge member of the spinal implant.

24. The insertion instrument of claim 22, further comprising a center rod operably coupled to the handle and to the jaws.

25. The insertion instrument of claim 24, wherein the center rod is slidably received within the shaft.

26. The insertion instrument of claim 22, wherein the jaws cooperate to define a centering member to cooperate with a centering portion of the spinal implant.

27. The insertion instrument of claim 17, wherein the handle includes channels for the receipt of a tool.

28. The insertion instrument of claim 27, wherein the handle includes an upper knob and a lower handle member, each of the upper knob and lower handle member including channels configured to be aligned with fasteners supported by the spinal implant when the spinal implant is gripped by the releasable gripping member.

29. An insertion instrument configured to manipulate a spinal implant, the insertion instrument comprising:
a handle;
a shaft coupled to the handle; and
an end effector coupled to the shaft and including a yoke rotatably supported on the shaft and configured to releasably couple to a fastener supported within a spinal implant device.

30. The insertion instrument of claim 29, wherein the end effector further includes a releasable gripping mechanism configured to releasably grip a spinal implant device.

31. The insertion instrument of claim 29, wherein the releasable gripping mechanism includes an retaining ring and a compressor ring operably coupled to the retaining ring.

32. The insertion instrument of claim 29, further comprising an actuator operably coupled to the shaft for transmitting energy to the end effector.

33. The insertion instrument of claim 29, wherein the releasable gripping member includes a pair of movable jaws configured to engage opposing lateral sides of the spinal implant.

34. The insertion instrument of claim 33, wherein each of the jaws include a tab that wraps around a bridge member of the spinal implant.

35. The insertion instrument of claim 29, wherein the handle includes channels for the receipt of a tool.

36. The insertion instrument of claim 29, further comprising a center rod operably coupled to the handle and to the jaws.

37. The insertion instrument of claim 36, wherein the center rod is slidably received within the shaft.

38. The insertion instrument of claim 29, wherein the jaws cooperate to define a centering member to cooperate with a centering portion of the spinal implant.

39. A surgical kit including:
a spinal implant including a bridge member and a pair of legs coupled to opposing ends of the bridge member;
an osteotome including a cutting member configured to precut bone for subsequent receipt of the legs of the spinal implant; and
an insertion instrument including a releasable gripping mechanism configured to releasably grip the spinal implant.

40. The spinal kit of claim 39, further comprising a pair of fasteners configured to secure the spinal implant to the bone, and a screw driver configured to cooperate with the fasteners.

41. The spinal kit of claim 39, wherein the osteotome further includes a gauge member having a planar profile with peripheral dimensions substantially identical to peripheral dimensions of the anterior-posterior planar profile of the spinal implant.

42. The spinal kit of claim 39, wherein the releasable gripping member includes a pair of movable jaws configured to engage opposing lateral sides of the spinal implant.

43. The spinal kit of claim 42, wherein each of the jaws include a tab that wraps around the bridge member of the spinal implant.

44. The spinal kit of claim 42, wherein the jaws cooperate to define a centering member to cooperate with a centering portion of the spinal implant.

45. A method of inserting a spinal implant comprising the steps of:
inserting a spinal implant into the spine using mechanical energy and simultaneously inserting an awl into the spine for precutting bone; and
inserting a fastener into an opening pre-cut by the awl.

46. The method of claim 45, further comprising the step of inserting a needle of a predetermined length into the spine to establish a datum prior to the step of inserting the spinal implant.

47. The method of claim 45, further comprising the steps of precutting bone prior to inserting the spinal implant, and inserting legs of the spinal implant into pre-cut openings.

48. The method of claim 45, wherein the step of inserting the spinal implant includes the step of releasably gripping a bridge member of the spinal implant.

49. A method of inserting a spinal implant comprising the steps of:
inserting chisel blades of an osteotome into the spine for pre-cutting bone and forming openings therein; and
inserting legs of a spinal implant into the openings pre-cut by the chisel blades.

50. The method of claim 49, further comprising the step of establishing a datum prior to the step of inserting legs of the spinal implant.

51. The method of claim 50, further comprising the step of selecting a proper size spinal implant by a plurality of different size osteotomes proximate the spine.

52. The method of claim 49, further comprising the step of inserting awls of the osteotome into the spine for pre-cutting bone and forming openings therein, and inserting fasteners into the openings pre-cut by the awls.

53. The method of claim 49, wherein the step of inserting legs of the spinal implant includes the step of releasably gripping a bridge member of the spinal implant.

54. The method of claim 49, further comprising the step of supporting fasteners within the spinal implant prior to the step of inserting legs of the spinal implant into the openings.

55. The method of claim 54, wherein the fasteners comprise screws and further comprising the steps of engaging the screws with a screwdriver and rotating the screws into engagement with the bone while holding the spinal implant in position with an insertion instrument.

56. The method of claim 49, further comprising the step of locating a centering portion of the spinal implant relative to a centering member of an insertion instrument.
